(19) United States
(12) Patent Application Publication
(10) Pub. No.: US 2006/0269514 A1
(43) Pub. Date: Nov. 30, 2006

(54) Methods and compositions for the modulation of immune responses and cancer diseases

(76) Inventor: Abdul-Rahman Jazieh, Mason, OH (US)

Correspondence Address: FROST BROWN TODD, LLC, 2200 PNC CENTER, 201 E. FIFTH STREET, CINCINNATI, OH, 45202, US

(21) Appl. No.: 11/140,102

(22) Filed: May 27, 2005

(51) Int. Cl. A61K 38/20; G01N 33/574

(52) U.S. Cl. 424/85.2; 435/7.23

(57) ABSTRACT

Disclosed is a method of treating, preventing or ameliorating disease using a chimeric protein comprising a cell-binding region and a biologically active region such as a toxin or other cytostatic or cytocidal molecule. The present invention discloses using a chimeric protein to modulate immune response to effectuate improvement in disease. In addition, the present invention teaches a method of targeting disease using the immune system. Finally, the present invention includes a method whereby the immune system may be used to monitor and assess disease progression, particularly with cancer diseases.

### FIELD OF THE INVENTION

The present invention relates to regulation of immune responses and cancer treatment. The present invention relates generally to methods of treating, preventing or ameliorating disease using a chimeric protein (polypeptide fusion protein) comprising of at least one biologically active peptide. More specifically, this invention relates to a method of treating, preventing or ameliorating disease using a chimeric protein comprised of at least of two portions. The first portion comprises a cell-binding/targeting portion that directs the protein to a particular cell type. The second portion comprises a therapeutic portion. The therapeutic portion may be in the form of a toxin or other cytostatic, cytocidal or other regulatory molecule. Upon binding of the chimeric protein to the target cell, the therapeutic portion comes in contact with the target cell where it exerts its regulatory or cytotoxic effect

### BACKGROUND OF THE INVENTION

There is a significant need in the art for a satisfactory treatment of cancer, and specifically lung, ovarian, breast, brain, colon and prostate cancers. Such a treatment could have a dramatic impact on the health of individuals, and especially older individuals, among whom cancer is especially common.

Hybrid proteins comprising a cell-binding portion and a toxin portion are known in the art. Typically, the cell-binding portion is a peptide that consists of a region of a ligand known to bind to a particular class of receptor. Toxins that have been used in combination with cell-binding peptides include ricin toxin A, diphtheria A, and pseudomonas exotoxin A.

Today's treatment of cancer remains, at best, only moderately successful. It is estimated that in 2004, there will be more than 1.2 million cases of cancer, and over 290,000 men and over 270,000 women will die of some form of cancer. In both sexes, lung cancer is the leading cause of death. Non-small-cell lung cancer, in particular, accounts for a significant proportion of lung cancer deaths. Chemotherapy treatment and surgical resection are currently available treatments for lung cancer, but survival rates are low, especially in cases of relapsed or refractory lung cancer.

Chimeric proteins (also referred to as “fusion proteins”) have surfaced as potential candidates for the treatment of disease. These molecules may be engineered to contain multiple biologically active segments, each having different, but specific effects. Hybrid proteins have been made that consist of a toxin molecule and a ligand that binds to cell surface receptors. The ligand portion directs the molecule to a particular class of cells where the toxin can exert its effect. There are many instances of chimeric proteins reported in the literature. For example, chimeric proteins consisting of a structural gene fused to a bacterial gene (Villa-Komaroff et al., Proc. Natl. Acad. Sci. U.S.A. 75: 3727-3731, 1978); therapeutic chimeric proteins consisting of a diphtheria toxin A chain coupled to a human placental lactogen hormone have been reported (Chang et al., J. Biol. Chem. 252:1515-1522, 1977); and, most pertinent to the present invention, Murphy, U.S. Pat. No. 6,022,950 teaches the use of chimeric molecules consisting of a cell-binding domain, a translocation domain (which transports the molecule across a cell membrane) and an “effector” domain that exerts a regulatory effect on the target cell. An additional feature taught in the literature is the use of “translocation domains” that facilitate transport of the protein across a cellular membrane. Translocation domains are used to stimulate the formation of endocytic vesicles that encapsulate the peptide and transport it into the cell where the therapeutic agent can exert its effect. (See Murphy, U.S. Pat. No. 6,022,950.)

The Murphy reference teaches the use of a chimeric protein that targets the interleukin II (IL-2) receptor. The IL2 receptor exists in three forms: low (CD25), intermediate (CD122/CD 132) and high (CD25/CD122/CD132) affinity. High affinity IL-2 receptors are usually found only on activated T-lymphocytes, activated B-lymphocytes and activated macrophages. IL-2 receptors have been reported on malignant cells of T and B cell origin in chronic lymphocytic leukemia, small lymphocytic lymphoma, cutaneous T-cell lymphoma, Hodgkin's disease and non-Hodgkin's lymphoma.

Despite the presence of IL2 receptors in many different cell types, IL-2 receptors do not exist in the high-affinity forms in lung tissue, suggesting limited use of an IL-2 chimeric protein to target lung cells. Of particular importance to the present invention, evidence suggests that the IL-2 receptor is found only in small numbers in non-small cell lung cancer cells (J Thorac Cardiovasc Surg. January 2000; 119(1):10-20). Thus, chimeric proteins bearing an IL-2 binding region would not be expected to bind to non-small cell lung cancer cells in significant amounts, and targeting IL-2 receptor bearing cells would not be expected to be successful in treating non-small cell lung cancer or other types of lung cancer.

In addition to the problem of finding an ideal target for chimeric protein therapy, the nature of solid tumors presents special problems in the use of any therapeutic agent. Effective treatment using a drug or other systemically administered therapeutic may be frustrated by the inability of the agent to penetrate a solid tumor. Compounds that cannot penetrate a solid cancer mass can only exert effect on a small number of cancer cells, severely limiting the effectiveness of a given agent. Because of this limitation, therapeutic agents must be given in higher doses for a longer period of time, resulting in increased expense, side effects and likelihood of failure. The present invention addresses these shortcomings of current treatments.

Finally, labeling of tumors can be helpful in disease management and monitoring of disease progression. Labeling agents such as florescent, radioactive, or electron-dense moieties can be used in combination with a cell-specific ligand to detect specific cell types. Murphy teaches combining a detectable label with a translocation region and a cell-binding ligand to achieve this effect. But, much like the physical limitations of therapeutic agents as discussed above, labeling agents are often limited in their ability to permeate a solid cancer. This again leads to the problems of increased expense and side effects of administering detectable labels to patients. In addition, ineffective detection methods lead to delayed treatment of disease and increased mortality rates. The present invention relates to a novel method of labeling and detecting cancer cells using circulating immune cells to permeate solid cancers.

ONTAK® (denileukin diftitox), a recombinant DNA-derived cytotoxic protein composed of the amino acid sequences for diphtheria toxin fragments A and B (Met₁-Thr₃₈₇)-His followed by the sequences for interleukin-2 (IL-2; Ala₁-Thr₁₃₃), is produced in an E. coli expression system.

ONTAK has a molecular weight of 58 kD. Neomycin is used in the fermentation process but is undetectable in the final product. The product is purified using reverse phase chromatography followed by a multistep diafiltration process. ONTAK is supplied in single use vials as a sterile, frozen solution intended for intravenous (IV) administration. Each 2 mL vial of ONTAK contains 300 mcg of recombinant denileukin diftitox in a sterile solution of citric acid (20 mM), EDTA (0.05 mM) and polysorbate 20 (<1%) in Water for Injection, USP. The solution has a pH of 6.9 to 7.2.

Denileukin diftitox is a fusion protein designed to direct the cytocidal action of diphtheria toxin to cells which express the IL-2 receptor. The human IL-2 receptor exists in three forms, low (CD25), intermediate (CD122/CD132) and high (CD25/CD122/CD132) affinity. The high affinity form of this receptor is usually found only on activated T lymphocytes, activated B lymphocytes and activated macrophages. Malignant cells expressing one or more of the subunits of the IL-2 receptor are found in certain leukemias and lymphomas including cutaneous T-cell lymphoma (CTCL)₁. Ex vivo studies suggest that denileukin diftitox interacts with the high affinity IL-2 receptor on the cell surface and inhibits cellular protein synthesis, resulting in cell death within hours. ONTAK is indicated for the treatment of patients with persistent or recurrent cutaneous T-cell lymphoma whose malignant cells express the CD25 component of the IL-2 receptor.

### BRIEF SUMMARY OF THE INVENTION

The present invention relates to regulation of immune responses and cancer treatment. The present invention relates generally to methods of treating, preventing or ameliorating disease using a chimeric protein (polypeptide fusion protein) comprising of at least one biologically active peptide. More specifically, this invention relates to a method of treating, preventing or ameliorating disease using a chimeric protein comprised of at least of two portions. The first portion comprises a cell-binding/targeting portion that directs the protein to a particular cell type. The second portion comprises a therapeutic portion. The therapeutic portion may be in the form of a toxin or other cytostatic, cytocidal or other regulatory molecule. Upon binding of the chimeric protein to the target cell, the therapeutic portion comes in contact with the target cell where it exerts its regulatory or cytotoxic effect.

This invention also relates generally to a method of treating, preventing or ameliorating disease via immune system modulation using chimeric proteins. Specifically, this invention relates to a method of targeting toxin or other inhibitory molecules to circulating immune cells which then carry the therapeutic molecule to target cells, systems or organs. More specifically, the invention relates to a method of targeting immune cells which then infiltrate solid cancers, thereby delivering the active substance to the core of the tumor, minimizing or eliminating the need to use agents that directly target the cancer.

Further, this invention relates generally to the use of a chimeric molecule consisting of a cell-binding portion and a labeling or marker portion. More specifically, this invention relates to an invention in which the cell-binding portion targets a class of circulating cells such that the chimeric protein bearing a detectable label can be targeted directly to a tumor. This method permits detection of tumor growth and location.

More specifically, this invention relates to a method of treating, preventing or ameliorating disease using a chimeric protein comprised of at least of two portions. The first portion consists essentially of a cell-binding/targeting portion that directs the protein to a particular cell type. The second portion consists essentially of a therapeutic portion. The therapeutic portion may be in the form of a toxin or other cytostatic, cytocidal or other regulatory molecule. Upon binding of the chimeric protein to the target cell, the therapeutic portion comes in contact with the target cell where it exerts its regulatory or cytotoxic effect.

The present invention also encompasses IL-2 polypeptide fusion proteins comprising a therapeutic protein (e.g., a polypeptide, antibody, or peptide, or fragments and variants thereof) fused to IL-2 polypeptide or a fragment (portion) or variant of IL-2 polypeptide. The present invention also encompasses IL-2 polypeptide fusion proteins comprising a therapeutic protein (e.g., a polypeptide, antibody, or peptide, or fragments and variants thereof) fused to IL-2 polypeptide or a fragment (portion) or variant of IL-2 polypeptide, that is sufficient to prolong the shelf life of the therapeutic protein, and/or stabilize the therapeutic protein and/or its activity in solution (or in a pharmaceutical composition) in vitro and/or in vivo. Nucleic acid molecules encoding the IL-2 polypeptide fusion proteins of the invention are also encompassed by the invention, as are vectors containing these nucleic acids, host cells transformed with these nucleic acids vectors, and methods of making the IL-2 polypeptide fusion proteins of the invention and using these nucleic acids, vectors, and/or host cells.

The invention also encompasses pharmaceutical formulations comprising an IL-2 polypeptide fusion protein of the invention and a pharmaceutically acceptable diluent or carrier. Such formulations may be in a kit or container. Such kit or container may be packaged with instructions pertaining to the extended shelf life of the therapeutic protein. Such formulations may be used in methods of treating, preventing, ameliorating or diagnosing a disease or disease symptom in a patient, preferably a mammal, most preferably a human, comprising the step of administering the pharmaceutical formulation to the patient.

The present compounds may also be used in co-therapies, partially or completely, in place of other conventional anti-tumor agents.

The present methods also include the sequential or concurrent administration of one or more additional therapeutics selected from the group comprising paclitaxel, etoposide, vinorelbine tartrate, carboplatin, docetaxel, Cisplatin, Gemcitabine, N(1),N(11)-diethylnorspermine (DENSPM), Gefitinib and Erlotinib.

These methods may employ the compounds of this invention in a monotherapy or in combination with an anti-tumor agent. Such combination therapies include administration of the agents in a single dosage form or in multiple dosage forms administered at the same time or at different times.

The above summary of the present invention is not intended to describe each embodiment or every implementation of the present invention. Advantages and attainments, together with a more complete understanding of the invention, will become apparent and appreciated by referring to the following detailed description and claims taken in conjunction with the accompanying drawings.

Throughout this document, all temperatures are given in degrees Celsius, and all percentages are weight percentages unless otherwise stated. All publications mentioned herein are incorporated herein by reference in their entirety for the purpose of describing and disclosing the compositions and methodologies, which are described in the publications which might be used in connection with the presently described invention. The publications discussed herein are provided solely for their disclosure prior to the filing date of the present application. Nothing herein is to be construed as an admission that the invention is not entitled to antedate such a disclosure by virtue of prior invention.

### DETAILED DESCRIPTION OF THE INVENTION

Before the present methods and compositions are described, it is to be understood that this invention is not limited to the specific methodology, devices, formulations, and compositions described as such may, of course, vary. It is also to be understood that the terminology used herein is for the purpose of describing particular embodiments only, and is not intended to limit the scope of the present invention, which will be limited only by the appended claims.

It must be noted that as used herein and in the appended claims, the singular forms “a”, “and”, and “the” include plural referents unless the context clearly dictates otherwise. Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

As used herein, the following terms shall have the definitions given below.

The term “administration” of the pharmaceutically active compounds and the pharmaceutical compositions defined herein includes systemic use, as by injection (especially parenterally), intravenous infusion, suppositories and oral administration thereof, as well as topical application of the compounds and compositions. Intravenous administration is particularly preferred in the present invention.

“Ameliorate” or “amelioration” means a lessening of the detrimental effect or severity of the cancer in the subject receiving therapy, the severity of the response being determined by means that are well known in the art.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer. The cancer to be treated is preferably cancerous growth of breast, ovary, prostate, lung, pancreas, and colorectal cells.

The terms “polypeptide” and “protein” refer to a polymer of amino acid residues and are not limited to a minimum length of the product. Thus, peptides, oligopeptides, dimers, multimers, and the like, are included within the definition. Both full-length proteins and fragments thereof are encompassed by the definition. The terms also include post-expression modifications of the polypeptide, for example, glycosylation, acetylation, phosphorylation and the like. Furthermore, for purposes of the present invention, a “polypeptide” refers to a protein which includes modifications, such as deletions, additions and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification. Thus, the term “IL-2 polypeptide” refers to native IL-2 sequences, as well as to IL-2 analogs, muteins and fragments, as defined further below.

By “purified” and “isolated” is meant, when referring to a polypeptide or polynucleotide, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated polynucleotide which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition.

The term “interleukin-2” or “IL-2” as used herein refers to a compound having the primary, secondary and/or tertiary molecular structure of native IL-2, and which has IL-2 activity as measured in standard IL-2 bioassays, such as the ability to stimulate proliferation of human IL-2 dependent cytolytic and helper T-cell lines (see, Gillis et al., J. Immunol. (1978) 120:2027-2032; Watson, J., J. exp. Med. (1979) 1570:1510-1519). The IL-2 molecule may include posttranslational modifications, such as glycosylation, acetylation, phosphorylation, etc. Furthermore, as ionizable amino and carboxyl groups are present in the molecule, a particular IL-2 may be obtained as an acidic or basic salt, or in neutral form. Additionally, for purposes of the present invention, an IL-2 polypeptide may be derived from any of several tissues of any mammalian source, such as human, bovine, murine, canine, equine, ovine, porcine, etc. The sequences for various IL-2 proteins are known.

The terms “IL-2 analog” and “IL-2 mutein” refer to biologically active derivatives of IL-2, or fragments of such derivatives, that retain IL-2 activity, as measured in assays as described above. In general, the term “analog” refers to compounds having a native polypeptide sequence and structure with one or more amino acid additions, substitutions (generally conservative in nature) and/or deletions, relative to the native molecule, so long as the modifications do not destroy IL-2 activity. The term “mutein” refers to peptides having one or more peptide mimics (“peptoids”), such as those described in International Publication No. WO 91/04282. Preferably, the analog or mutein has at least the same activity as the native molecule. Methods for making polypeptide analogs and muteins are known in the art and are described further below.

Particularly preferred analogs include substitutions that are conservative in nature, i.e., those substitutions that take place within a family of amino acids that are related in their side chains. Specifically, amino acids are generally divided into four families: (1) acidic-aspartate and glutamate; (2) basic-lysine, arginine, histidine; (3) non-polar-alanine, valine, leucine, isoleucine, proline, phenylalaninc, methionine, tryptophan; and (4) uncharged polar-glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity.

By “fragment” is intended a polypeptide consisting of only a part of the intact polypeptide sequence and structure. The fragment can include a C-terminal deletion or N-terminal deletion of the native polypeptide. A “fragment” of IL-2 will generally include at least about 10 contiguous amino acid residues of the full-length molecule, preferably at least about 15-25 contiguous amino acid residues of the full-length molecule, and most preferably at least about 20-50 or more contiguous amino acid residues of full-length IL-2, or any integer between 10 amino acids and the full-length sequence, provided that the fragment in question retains IL-2 activity as described above. One preferred fragment of IL-2 is a molecule having one or more of the first five N-terminal amino acids of the native IL-2 molecule deleted.

By “recombinant IL-2” is intended an IL-2 molecule having biological activity, as measured using the techniques described above and which has been prepared by recombinant DNA techniques as described herein. See, also, for example, Taniguchi, et al. Nature (1983) 302:305-310 and Devos Nucleic Acids Research (1983) 11:4307-4323 or mutationally altered IL-2 as described by Wang, et al. Science (1984) 224:1431-1433. In general, the gene coding for IL-2 is cloned and then expressed in transformed organisms, as described further below. The host organism expresses the foreign gene to produce IL-2 under expression conditions. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,298; and 4,931,543, herein incorporated by reference in their entireties.

A “specific binding molecule” intends a molecule that, through chemical or physical means, specifically binds to a second molecule. One example of a specific binding molecule is an antibody molecule. The term “antibody” as used herein includes antibodies obtained from both polyclonal and monoclonal preparations, as well as, the following: hybrid (chimeric) antibody molecules (see, for example, Winter et al. (1991) Nature 349:293-299; and U.S. Pat. No. 4,816,567); F(ab′).sub.2 and F(ab) fragments; Fv molecules (non-covalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, for example, Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B: 120-126); humanized antibody molecules (see, for example, Riechmann et al. (1988) Nature 332:323-327; Verhoeyanetal. (1988) Science 239:1534-1536; and U.K. Patent Publication No. GB 2,276,169, published Sep. 21, 1994); and, any functional fragments obtained from such molecules, wherein such fragments retain immunological binding properties of the parent antibody molecule.

As used herein, the term “monoclonal antibody” refers to an antibody composition having a homogeneous antibody population. The term is not limited regarding the species or source of the antibody, nor is it intended to be limited by the manner in which it is made. Thus, the term encompasses antibodies obtained from murine hybridomas, as well as human monoclonal antibodies obtained using human rather than murine hybridomas. See, e.g., Cote, et al. Monoclonal Antibodies and Cancer Therapy, Alan R. Liss, 1985, p. 77.

An IL-2 polypeptide is “linked” to a therapeutic protein when the IL-2 polypeptide is chemically coupled to, or associated with the specific binding molecule, or when expressed from a chimeric polynucleotide which encodes the IL-2 polypeptide and the therapeutic protein of interest. The term “linked” intends that the IL-2 polypeptide may either be directly linked to the therapeutic protein or may be linked via a linker moiety, such as via a peptide linker described below.

An “immunoconjugate” or “fusion” is an IL-2 polypeptide which is linked to a therapeutic protein, as defined above. Thus, the term denotes both chemically conjugated as well as recombinantly produced fusion molecules.

As used herein, the terms “treating” or “treatment” of a disease include preventing the disease, i.e. preventing clinical symptoms of the disease in a subject that may be exposed to, or predisposed to, the disease, but does not yet experience or display symptoms of the disease; inhibiting the disease, i.e., arresting the development of the disease or its clinical symptoms, such as by suppressing tumor cell growth; or relieving the disease, i.e., causing regression of the disease or its clinical symptoms.

The terms “effective amount” or “pharmaceutically effective amount” refer to a nontoxic but sufficient amount of the agent to provide the desired biological result. That result can be reduction and/or alleviation of the signs, symptoms, or causes of a disease, such as cancer, or any other desired alteration of a biological system. Such amounts are described below. An appropriate “effective” amount in any individual case may be determined by one of ordinary skill in the art using routine experimentation.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals and non-mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or sex.

The present invention relates generally to polypeptide fusion proteins and methods of treating, preventing, or ameliorating diseases or disorders. Preferably, the chimeric molecule is one as described by U.S. Pat. No. 6,022,950, incorporated herein by reference in its entirety. More preferably the molecule is Denileukin diftitox.

Denileukin diftitox is a recombinant DNA-derived cytotoxic protein composed of the amino acid sequence for diphtheria toxin fragments and the sequence for interleukin-2 (IL-2). The ability of the compound to interact with IL-2 receptor bearing cells allows it to target the toxin directly to cells having that receptor. IL-2 receptors exist in three forms: low, intermediate and high affinity. The compound interacts with high affinity IL-2 receptors on cell surfaces, causing cell death within hours. High affinity IL-2 receptors are usually found only on activated T lymphocytes, activated B lymphocytes and activated macrophages.

Without wishing to be bound by theory, the precise mechanism by which the compound achieves its cytocidal effects on tumors is uncertain, several theories are herein proposed.

In one embodiment, the compound has a direct “immune modulation” effect. By interacting with circulating lymphocytes that have an inhibitory effect on the immune response, the cytocidal property of the compound inhibits immunosuppressant cells, thereby increasing the overall immune response. This enhanced immunity then results in improved resistance to tumor growth and destruction of cancer cells, without the compound interacting directly with the tumor cells.

In a second embodiment, the circulating lymphocytes and macrophages act as carriers for the compound into established tumors. Studies indicate that immune cells (particularly B lymphocytes and macrophages) infiltrate tumors during the immune response. In addition to directly interacting with IL-2 receptors on the surface of tumor cells, the compound also interacts with and binds to circulating immune cells that then infiltrate the tumor. This infiltration of compound-carrying immune cells permits the compound to be directly targeted to the tumor cells, where the toxin can exert its effect. Thus, in addition to targeting tumor cells that bear the IL-2 receptor, tumors that are infiltrated by IL-2 receptor bearing immune cells are also targeted. The potential uses for the compound include uses for many types of cancers that would not ordinarily have high-affinity IL-2 receptors.

The present invention relates generally to IL-2 polypeptide fusion proteins and methods of treating, preventing, or ameliorating diseases or disorders. As used herein, “IL-2 polypeptide fusion protein” refers to a protein formed by the fusion of at least one molecule of IL-2 polypeptide (or a fragment or variant thereof) to at least one molecule of a therapeutic protein (or fragment or variant thereof). An IL-2 polypeptide fusion protein of the invention comprises at least a fragment or variant of a therapeutic protein and at least a fragment or variant of human serum IL-2 polypeptide, which are associated with one another, preferably by genetic fusion (i.e., the IL-2 polypeptide fusion protein is generated by translation of a nucleic acid in which a polynucleotide encoding all or a portion of a therapeutic protein is joined in-frame with a polynucleotide encoding all or a portion of IL-2 polypeptide) or chemical conjugation to one another. The therapeutic protein and IL-2 polypeptide protein, once part of the IL-2 polypeptide fusion protein, may be referred to as a “portion”, “region” or “moiety” of the IL-2 polypeptide fusion protein (e.g., a “therapeutic protein portion” or an “IL-2 polypeptide protein portion”).

In one embodiment, the invention provides an IL-2 polypeptide fusion protein comprising, or alternatively consisting of, a therapeutic protein (e.g., as described in Table 1) and a serum IL-2 polypeptide protein. In other embodiments, the invention provides an IL-2 polypeptide fusion protein comprising, or alternatively consisting of, a biologically active and/or therapeutically active fragment of a therapeutic protein and a serum IL-2 polypeptide protein. In other embodiments, the invention provides an IL-2 polypeptide fusion protein comprising, or alternatively consisting of, a biologically active and/or therapeutically active variant of a therapeutic protein and a serum IL-2 polypeptide protein. In preferred embodiments, the serum IL-2 polypeptide protein component of the IL-2 polypeptide fusion protein is the mature portion of serum IL-2 polypeptide.

In further embodiments, the invention provides an IL-2 polypeptide fusion protein comprising, or alternatively consisting of, a therapeutic protein, and a biologically active and/or therapeutically active fragment of serum IL-2 polypeptide. In further embodiments, the invention provides an IL-2 polypeptide fusion protein comprising, or alternatively consisting of, a therapeutic protein and a biologically active and/or therapeutically active variant of serum IL-2 polypeptide. In preferred embodiments, the therapeutic protein portion of the IL-2 polypeptide fusion protein is the mature portion of the therapeutic protein. In a further preferred embodiment, the therapeutic protein portion of the IL-2 polypeptide fusion protein is the extracellular soluble domain of the therapeutic protein. In an alternative embodiment, the therapeutic protein portion of the IL-2 polypeptide fusion protein is the active form of the therapeutic protein.

In further embodiments, the invention provides an IL-2 polypeptide fusion protein comprising, or alternatively consisting of, a biologically active and/or therapeutically active fragment or variant of a therapeutic protein and a biologically active and/or therapeutically active fragment or variant of serum IL-2 polypeptide. In preferred embodiments, the invention provides an IL-2 polypeptide fusion protein comprising, or alternatively consisting of, the mature portion of a therapeutic protein and the mature portion of serum IL-2 polypeptide.

In general, the present invention encompasses a composition comprised of a hybrid molecule that includes a first part and a second part connected by a covalent bond. The first part is an amino acid sequence that consists of a cell-binding region, said cell-binding region selected from the group consisting of a portion of a hormone, a single chain analog of a monoclonal antibody, or a portion of a polypeptide chain. The second part is a chemically biologically active molecule. The second part may be a toxin selected from the group consisting of diphtheria toxin, Pseudomonas exotoxin A, cholera toxin, ricin toxin, and Shiga-like toxin. Alternatively, the second part may be a detectable label selected from the group consisting of ¹²⁵I-compounds, technetium isotopes, NMR reporter groups, and fluorescent dyes.

In one embodiment, this invention relates to a method of treating, preventing or ameliorating disease using a chimeric protein comprised of at least of two portions. The first portion consists essentially of a cell-binding/targeting portion that directs the protein to a particular cell type. The second portion consists essentially of a therapeutic portion. The therapeutic portion may be in the form of a toxin or other cytostatic, cytocidal or other regulatory molecule. Upon binding of the chimeric protein to the target cell, the therapeutic portion comes in contact with the target cell where it exerts its regulatory or cytotoxic effect.

The hybrid molecule may also contain a translocation region that facilitates transporting all or part of the chimeric protein into the cell. The translocation domain may be one as described by Murphy, U.S. Pat. No. 6,022,950. The molecule may also contain a peptide sequence that initiates cleavage of the chimeric protein. Preferably, the described cell-binding region is one that binds to a receptor expressed on circulating immune cells. More preferably, the receptor is IL-2 . More preferably, the cell-binding region is that described by U.S. Pat. No. 6,022,950. More preferably, the compound is denileukin diftitox.

In addition to the molecule described above, the present invention relates to a method of treating, preventing or ameliorating disease using the above-described compound. In one embodiment, the first part of the chimeric protein described above is a portion of a ligand that binds to a circulating immune cell. The immune cell is selected from the group consisting of macrophages, B-lymphocytes, or T-lymphocytes. In this embodiment of the present invention, chimeric proteins are used to target circulating immune cells. While the precise mechanism of action by which the compound achieves its cytocidal effects on tumors is uncertain, several theories are herein proposed.

First, the compound may have a direct “immune modulation” effect. Under this theory, the regulatory portion of the chimeric protein inhibits with circulating lymphocytes that suppress immune system function. By inhibiting immuno-suppressant cells, the compound effectively increases the overall immune response. This enhanced immunity then results in improved resistance to tumor growth and destruction of cancer cells, without the compound interacting directly with the tumor cells.

Alternatively, the circulating lymphocytes and macrophages act as carriers for the compound into established tumors. Studies indicate that immune cells (particularly B lymphocytes and macrophages) infiltrate tumors during the immune response. In addition to directly interacting with IL-2 receptors on the surface of tumor cells, the compound also binds to circulating immune cells that then infiltrate the tumor. This infiltration of compound-carrying immune cells permits the compound to be directly targeted to the tumor cells, particularly the interior of solid cancers, where the toxin can exert its effect. This mechanism allows for more effective use of toxic compounds, minimizing expense and side effects of treatment. In addition, this method expands the potential targets for existing compounds. By using receptors that exist on the many types of circulating immune cells, the pool of target sites is now greatly expanded.

At present, the types of receptors expressed on a cancerous cell may limit chimeric protein therapy for cancer. This method allows the use of other, known receptors on circulating immune cells to be used as potential targets. For example, the known compound denileukin diftitox would be expected to have limited application to lung cancers which have little or no expression of the IL-2 receptor. However, example 1 (below) demonstrates that denileukin diftitox is effective in treating non-small-cell lung cancer. This is an unexpected result because of the limited number of IL-2 receptors in this type of tissue. Thus, the potential uses for the compound include uses for many types of cancers that would not ordinarily have high-affinity IL-2 receptors.

In another embodiment, the present invention can be used to treat lung cancer such as non-small cell lung cancer. Preferably, the chimeric molecule is one as described by U.S. Pat. No. 6,022,950, incorporated herein by reference in its entirety. More preferably the molecule is Denileukin diftitox.

Denileukin diftitox is a recombinant DNA-derived cytotoxic protein composed of the amino acid sequence for diphtheria toxin fragments and the sequence for interleukin-2 (IL-2). The ability of the compound to interact with IL-2 receptor bearing cells allows it to target the toxin directly to cells having that receptor. IL-2 receptors exist in three forms: low, intermediate and high affinity. The compound interacts with high affinity IL-2 receptors on cell surfaces, causing cell death within hours. High affinity IL-2 receptors are usually found only on activated T-lymphocytes, activated B-lymphocytes and activated macrophages.

High affinity IL-2 receptors are not found in lung tissues. In addition, all forms of IL-2 receptors are found in small amounts in lung tissue. In particular, non-small cell lung cancers have not been found to express significant amounts of the IL-2 receptor. Because of this fact, a chimeric protein having an IL-2 receptor binding region would not be expected to bind to lung cancers in general or non-small cell lung cancer in particular. Thus, the chimeric protein denileukin diftitox as described in U.S. Pat. No. 6,022,950 would not be expected to be efficacious in treating non-small cell lung cancer.

However, as described in example 1, a chimeric protein comprised of an IL-2 cell-binding portion and a cytotoxic portion is effective in treating non-small cell lung cancer. With respect to this embodiment of the invention, all methods of making a chimeric protein as described in U.S. Pat. No. 6,022,950 are incorporated herein by reference.

In another embodiment, the present invention may be used to monitor response to treatment in an individual with lung cancer, comprising administering an effective amount of a chimeric protein to the individual and measuring soluble interleukin-2 receptor levels of the individual. Preferably, in this embodiment, the cell-binding portion of the chimeric protein is analogous to the IL-2 receptor. More preferably, the molecule is denileukin diftitox.

In yet another embodiment, the present invention may be used to detect, diagnose or monitor disease. In this embodiment, the first portion of the chimeric protein consists of a cell binding region analogous to a ligand that binds to receptors expressed on circulating immune cells such as b-cells, t-cells, macrophages or lymphocytes. The second portion of the chimeric protein is a detectable label selected from the group consisting of ¹²⁵I-compounds, technetium isotopes, NMR reporter groups and fluorescent dyes. In this embodiment, the chimeric protein is targeted to the circulating immune cells that then carry the detectable label into a solid tumor by infiltration. Alternatively, the chimeric protein may bind to circulating immune cells that have already infiltrated a solid tumor.

Therapeutic Proteins

As stated above, an IL-2 polypeptide fusion protein of the invention comprises at least a fragment or variant of a therapeutic protein and at least a fragment or variant of human serum IL-2 polypeptide, which are associated with one another, preferably by genetic fusion or chemical conjugation.

As used herein, “therapeutic protein” refers to proteins, polypeptides, antibodies, peptides or fragments or variants thereof, having one or more therapeutic and/or biological activities. therapeutic proteins encompassed by the invention include but are not limited to, proteins, polypeptides, peptides, antibodies, and biologics. (The terms peptides, proteins, and polypeptides are used interchangeably herein.) It is specifically contemplated that the term “therapeutic protein” encompasses antibodies and fragments and variants thereof. Thus an IL-2 polypeptide fusion protein of the invention may contain at least a fragment or variant of a therapeutic protein, and/or at least a fragment or variant of an antibody. Additionally, the term “therapeutic protein” may refer to the endogenous or naturally occurring correlate of a therapeutic protein.

By a polypeptide displaying a “therapeutic activity” or a protein that is “therapeutically active” is meant a polypeptide that possesses one or more known biological and/or therapeutic activities associated with a therapeutic protein such as one or more of the therapeutic proteins described herein or otherwise known in the art. As a non-limiting example, a “therapeutic protein” is a protein that is useful to treat, prevent or ameliorate a disease, condition or disorder. As a non-limiting example, a “therapeutic protein” may be one that binds specifically to a particular cell type (normal (e.g., lymphocytes) or abnormal e.g., (cancer cells)) and therefore may be used to target a compound (drug, or cytotoxic agent) to that cell type specifically.

In another non-limiting example, a “therapeutic protein” is a protein that has a biological activity, and in particular, a biological activity that is useful for treating preventing or ameliorating a disease. A non-inclusive list of biological activities that may be possessed by a therapeutic protein includes, enhancing the immune response, promoting angiogenesis, inhibiting angiogenesis, regulating hematopoietic functions, stimulating nerve growth, enhancing an immune response, inhibiting an immune response, or any one or more of the biological activities described in the “Biological Activities” section below.

Therapeutic proteins corresponding to a therapeutic protein portion of an IL-2 polypeptide fusion protein of the invention, such as cell surface and secretory proteins, are often modified by the attachment of one or more oligosaccharide groups. The modification, referred to as glycosylation, can dramatically affect the physical properties of proteins and can be important in protein stability, secretion, and localization. Glycosylation occurs at specific locations along the polypeptide backbone. There are usually two major types of glycosylation: glycosylation characterized by O-linked oligosaccharides, which are attached to serine or threonine residues; and glycosylation characterized by N-linked oligosaccharides, which are attached to asparagine residues in an Asn-X-Ser/Thr sequence, where X can be any amino acid except proline. N-acetylneuramic acid (also known as sialic acid) is usually the terminal residue of both N-linked and O-linked oligosaccharides. Variables such as protein structure and cell type influence the number and nature of the carbohydrate units within the chains at different glycosylation sites. Glycosylation isomers are also common at the same site within a given cell type.

Therapeutic proteins corresponding to a therapeutic protein portion of an IL-2 polypeptide fusion protein of the invention, as well as analogs and variants thereof, may be modified so that glycosylation at one or more sites is altered as a result of manipulation(s) of their nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that will not glycosylate them, e.g. in E. coli or glycosylation-deficient yeast. These approaches are described in more detail below and are known in the art.

Therapeutic proteins corresponding to a therapeutic protein portion of an IL-2 polypeptide fusion protein of the invention include, but are not limited to, calcitonin, growth hormone releasing factor, insulin-like growth factor-1, interferon beta, interleukin-2, and parathyroid hormone,. These proteins and nucleic acid sequences encoding these proteins are well known and available in public databases such as Chemical Abstracts Services Databases (e.g., the CAS Registry), GenBank, and GenSeq.

Polypeptide and Polynucleotide Fragments and Variants

Fragments

Even if deletion of one or more amino acids from the N-terminus of a protein results in modification or loss of one or more biological functions of the therapeutic protein, IL-2 polypeptide protein, and/or IL-2 polypeptide fusion protein, other Therapeutic activities and/or functional activities (e.g., biological activities, ability to multimerize, ability to bind a ligand) may still be retained. For example, the ability of polypeptides with N-terminal deletions to induce and/or bind to antibodies which recognize the complete or mature forms of the polypeptides generally will be retained when less than the majority of the residues of the complete polypeptide are removed from the N-terminus. Whether a particular polypeptide lacking N-terminal residues of a complete polypeptide retains such immunologic activities can readily be determined by routine methods described herein and otherwise known in the art. It is not unlikely that a mutein with a large number of deleted N-terminal amino acid residues may retain some biological or immunogenic activities. In fact, peptides composed of as few as six amino acid residues may often evoke an immune response.

Accordingly, fragments of a therapeutic protein corresponding to a therapeutic protein portion of an IL-2 polypeptide fusion protein of the invention, include the full length protein as well as polypeptides having one or more residues deleted from the amino terminus of the amino acid sequence of the reference polypeptide (e.g., a therapeutic protein as disclosed in Table 1). In particular, N-terminal deletions may be described by the general formula m-q, where q is a whole integer representing the total number of amino acid residues in a reference polypeptide, and m is defined as any integer ranging from 2 to q-6. Polynucleotides encoding these polypeptides are also encompassed by the invention.

In addition, fragments of serum IL-2 polypeptide polypeptides corresponding to an IL-2 polypeptide protein portion of an IL-2 polypeptide fusion protein of the invention, include the full length protein as well as polypeptides having one or more residues deleted from the amino terminus of the amino acid sequence of the reference polypeptide (i.e., serum IL-2 polypeptide). Moreover, fragments of IL-2 polypeptide fusion proteins of the invention, include the full length IL-2 polypeptide fusion protein as well as polypeptides having one or more residues deleted from the amino terminus of the IL-2 polypeptide fusion protein. In particular, N-terminal deletions may be described by the general formula m-q, where q is a whole integer representing the total number of amino acid residues in the IL-2 polypeptide fusion protein, and m is defined as any integer ranging from 2 to q-6. Polynucleotides encoding these polypeptides are also encompassed by the invention.

Also as mentioned above, even if deletion of one or more amino acids from the N-terminus or C-terminus of a reference polypeptide (e.g., a therapeutic protein and/or serum IL-2 polypeptide protein) results in modification or loss of one or more biological functions of the protein, other functional activities (e.g., biological activities, ability to multimerize, ability to bind a ligand) and/or Therapeutic activities may still be retained. For example the ability of polypeptides with C-terminal deletions to induce and/or bind to antibodies which recognize the complete or mature forms of the polypeptide generally will be retained when less than the majority of the residues of the complete or mature polypeptide are removed from the C-terminus. Whether a particular polypeptide lacking the N-terminal and/or C-terminal residues of a reference polypeptide retains Therapeutic activity can readily be determined by routine methods described herein and/or otherwise known in the art.

The present invention further provides polypeptides having one or more residues deleted from the carboxy terminus of the amino acid sequence of a therapeutic protein corresponding to a therapeutic protein portion of an IL-2 polypeptide fusion protein of the invention (e.g., a therapeutic protein referred to in Table 1). In particular, C-terminal deletions may be described by the general formula 1-n, where n is any whole integer ranging from 6 to q-1, and where q is a whole integer representing the total number of amino acid residues in a reference polypeptide. Polynucleotides encoding these polypeptides are also encompassed by the invention.

Variants

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant”, refers to a therapeutic protein portion of an IL-2 polypeptide fusion protein of the invention, IL-2 polypeptide portion of an IL-2 polypeptide fusion protein of the invention, or IL-2 polypeptide fusion protein differing in sequence from a therapeutic protein (e.g. see “therapeutic” column of Table 1), IL-2 polypeptide protein, and/or IL-2 polypeptide fusion protein of the invention, respectively, but retaining at least one functional and/or therapeutic property thereof (e.g., a therapeutic activity and/or biological activity as disclosed in the “Biological Activity” column of Table 1) as described elsewhere herein or otherwise known in the art. Generally, variants are overall very similar, and, in many regions, identical to the amino acid sequence of the therapeutic protein corresponding to a therapeutic protein portion of an IL-2 polypeptide fusion protein of the invention, IL-2 polypeptide protein corresponding to an IL-2 polypeptide protein portion of an IL-2 polypeptide fusion protein of the invention, and/or IL-2 polypeptide fusion protein of the invention. Nucleic acids encoding these variants are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 95%, 96%, 97%, 98% or 99% identical to, for instance, the amino acid sequence of an IL-2 polypeptide fusion protein of the invention or a fragment thereof (such as the therapeutic protein portion of the IL-2 polypeptide fusion protein or the IL-2 polypeptide portion of the IL-2 polypeptide fusion protein), can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Biosci. 6:237-245 (1990)). In a sequence alignment the query and subject sequences are either both nucleotide sequences or both amino acid sequences. The result of said global sequence alignment is expressed as percent identity. Preferred parameters used in a FASTDB amino acid alignment are: Matrix=PAM 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject amino acid sequence, whichever is shorter.

If the subject sequence is shorter than the query sequence due to N— or C-terminal deletions, not because of internal deletions, a manual correction must be made to the results. This is because the FASTDB program does not account for N— and C-terminal truncations of the subject sequence when calculating global percent identity. For subject sequences truncated at the N— and C-termini, relative to the query sequence, the percent identity is corrected by calculating the number of residues of the query sequence that are N— and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N— and C-termini of the subject sequence, which are not matched/aligned with the query sequence, are considered for the purposes of manually adjusting the percent identity score. That is, only query residue positions outside the farthest N— and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a matching/alignment of the first 10 residues at the N— terminus. The 10 unpaired residues represent 10% of the sequence (number of residues at the N— and C-termini not matched/total number of residues in the query sequence) so 10% is subtracted from the percent identity score calculated by the FASTDB program. If the remaining 90 residues were perfectly matched the final percent identity would be 90%. In another example, a 90 residue subject sequence is compared with a 100 residue query sequence. This time the deletions are internal deletions so there are no residues at the N— or C-termini of the subject sequence which are not matched/aligned with the query. In this case the percent identity calculated by FASTDB is not manually corrected. Once again, only residue positions outside the N— and C-terminal ends of the subject sequence, as displayed in the FASTDB alignment, which are not matched/aligned with the query sequence, are manually corrected for. No other manual corrections are made for the purposes of the present invention.

The variant will usually have at least 75% (preferably at least about 80%, 90%, 95% or 99%) sequence identity with a length of normal HA or therapeutic protein which is the same length as the variant. Homology or identity at the nucleotide or amino acid sequence level is determined by BLAST (Basic Local Alignment Search Tool) analysis using the algorithm employed by the programs blastp, blastn, blastx, tblastn and tblastx (Karlin et al., Proc. Natl. Acad. Sci. USA 87: 2264-2268 (1990) and Altschul, J. Mol. Evol. 36: 290-300 (1993), fully incorporated by reference) which are tailored for sequence similarity searching.

The approach used by the BLAST program is to first consider similar segments between a query sequence and a database sequence, then to evaluate the statistical significance of all matches that are identified and finally to summarize only those matches which satisfy a pre-selected threshold of significance. For a discussion of basic issues in similarity searching of sequence databases, see Altschul et al., (Nature Genetics 6: 119-129 (1994)) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., Proc. Natl. Acad. Sci. USA 89: 10915-10919 (1992), fully incorporated by reference). For blastn, the scoring matrix is set by the ratios of M (i.e., the reward score for a pair of matching residues) to N (i.e., the penalty score for mismatching residues), wherein the default values for M and N are 5 and −4, respectively. Four blastn parameters may be adjusted as follows: Q=10 (gap creation penalty); R=10 (gap extension penalty); wink=1 (generates word hits at every wink^{th} position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

The polynucleotide variants of the invention may contain alterations in the coding regions, non-coding regions, or both. Especially preferred are polynucleotide variants containing alterations which produce silent substitutions, additions, or deletions, but do not alter the properties or activities of the encoded polypeptide. Nucleotide variants produced by silent substitutions due to the degeneracy of the genetic code are preferred. Moreover, polypeptide variants in which less than 50, less than 40, less than 30, less than 20, less than 10, or 5-50, 5-25, 5-10, 1-5, or 1-2 amino acids are substituted, deleted, or added in any combination are also preferred. Polynucleotide variants can be produced for a variety of reasons, e.g., to optimize codon expression for a particular host (change codons in the human mRNA to those preferred by a bacterial host, such as, yeast or E. coli).

In a preferred embodiment, a polynucleotide encoding an IL-2 polypeptide portion of an IL-2 polypeptide fusion protein of the invention is optimized for expression in yeast or mammalian cells. In further preferred embodiment, a polynucleotide encoding a therapeutic protein portion of an IL-2 polypeptide fusion protein of the invention is optimized for expression in yeast or mammalian cells. In a still further preferred embodiment, a polynucleotide encoding an IL-2 polypeptide fusion protein of the invention is optimized for expression in yeast or mammalian cells.

In an alternative embodiment, a codon optimized polynucleotide encoding a therapeutic protein portion of an IL-2 polypeptide fusion protein of the invention does not hybridize to the wild type polynucleotide encoding the therapeutic protein under stringent hybridization conditions as described herein. In a further embodiment, a codon optimized polynucleotide encoding an IL-2 polypeptide portion of an IL-2 polypeptide fusion protein of the invention does not hybridize to the wild type polynucleotide encoding the IL-2 polypeptide protein under stringent hybridization conditions as described herein. In another embodiment, a codon optimized polynucleotide encoding an IL-2 polypeptide fusion protein of the invention does not hybridize to the wild type polynucleotide encoding the therapeutic protein portion or the IL-2 polypeptide protein portion under stringent hybridization conditions as described herein.

In an additional embodiment, polynucleotides encoding a therapeutic protein portion of an IL-2 polypeptide fusion protein of the invention do not comprise, or alternatively consist of, the naturally occurring sequence of that therapeutic protein. In a further embodiment, polynucleotides encoding an IL-2 polypeptide protein portion of an IL-2 polypeptide fusion protein of the invention do not comprise, or alternatively consist of, the naturally occurring sequence of IL-2 polypeptide protein. In an alternative embodiment, polynucleotides encoding an IL-2 polypeptide fusion protein of the invention do not comprise, or alternatively consist of, the naturally occurring sequence of a therapeutic protein portion or the IL-2 polypeptide protein portion.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. See Pinckard et al., Clin. Exp. Immunol. 2:331-340 (1967); Robbins et al., Diabetes 36: 838-845 (1987); Cleland et al., Crit. Rev. Therapeutic Drug Carrier Systems 10:307-377 (1993).

In specific embodiments, the polypeptides of the invention comprise, or alternatively, consist of, fragments or variants of the amino acid sequence of a therapeutic protein described herein and/or human serum IL-2 polypeptide, and/or IL-2 polypeptide fusion protein of the invention, wherein the fragments or variants have 1-5, 5-10, 5-25, 5-50, 10-50 or 50-150, amino acid residue additions, substitutions, and/or deletions when compared to the reference amino acid sequence. In preferred embodiments, the amino acid substitutions are conservative. Nucleic acids encoding these polypeptides are also encompassed by the invention.

Functional Activity

“A polypeptide having functional activity” refers to a polypeptide capable of displaying one or more known functional activities associated with the full-length, pro-protein, and/or mature form of a therapeutic protein. Such functional activities include, but are not limited to, biological activity, antigenicity (the ability to bind, or compete with a polypeptide for binding, to an anti-polypeptide antibody), immunogenicity (ability to generate antibody, which binds to a specific polypeptide of the invention), ability to form multimers with polypeptides of the invention, and ability to bind to a receptor or ligand for a polypeptide.

“A polypeptide having biological activity” refers to a polypeptide exhibiting activity similar to, but not necessarily identical to, an activity of a therapeutic protein of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention (i.e., the candidate polypeptide will exhibit greater activity or not more than about 25-fold less and, preferably, not more than about tenfold less activity, and most preferably, not more than about three-fold less activity relative to the polypeptide of the present invention).

In preferred embodiments, an IL-2 polypeptide fusion protein of the invention has at least one biological and/or therapeutic activity associated with the therapeutic protein (or fragment or variant thereof) when it is not fused to IL-2 polypeptide.

The IL-2 polypeptide fusion proteins of the invention can be assayed for functional activity (e.g., biological activity) using or routinely modifying assays known in the art, as well as assays described herein. Specifically, IL-2 polypeptide fusion proteins may be assayed for functional activity (e.g., biological activity or therapeutic activity) using the assay referenced in the “Exemplary Activity Assay” column of Table 1. Additionally, one of skill in the art may routinely assay fragments of a therapeutic protein corresponding to a therapeutic protein portion of an IL-2 polypeptide fusion protein of the invention, for activity using assays referenced in its corresponding row of Table 1. Further, one of skill in the art may routinely assay fragments of an IL-2 polypeptide protein corresponding to an IL-2 polypeptide protein portion of an IL-2 polypeptide fusion protein of the invention, for activity using assays known in the art and/or as described in the Examples section below.

For example, in one embodiment where one is assaying for the ability of an IL-2 polypeptide fusion protein of the invention to bind or compete with a therapeutic protein for binding to an anti-Therapeutic polypeptide antibody and/or anti-IL-2 polypeptide antibody, various immunoassays known in the art can be used, including but not limited to, competitive and non-competitive assay systems using techniques such as radioimmunoassays, ELISA (enzyme linked immunosorbent assay), “sandwich” immunoassays, immunoradiometric assays, gel diffusion precipitation reactions, immunodiffusion assays, in situ immunoassays (using colloidal gold, enzyme or radioisotope labels, for example), western blots, precipitation reactions, agglutination assays (e.g., gel agglutination assays, hemagglutination assays), complement fixation assays, immunofluorescence assays, protein A assays, and immunoelectrophoresis assays, etc. In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention.

In a preferred embodiment, where a binding partner (e.g., a receptor or a ligand) of a therapeutic protein is identified, binding to that binding partner by an IL-2 polypeptide fusion protein containing that therapeutic protein as the therapeutic protein portion of the fusion can be assayed, e.g., by means well-known in the art, such as, for example, reducing and non-reducing gel chromatography, protein affinity chromatography, and affinity blotting. See generally, Phizicky et al., Microbiol. Rev. 59:94-123 (1995). In another embodiment, the ability of physiological correlates of an IL-2 polypeptide fusion protein of the present invention to bind to a substrate(s) of the Therapeutic polypeptide corresponding to the Therapeutic portion of the IL-2 polypeptide fusion protein of the invention can be routinely assayed using techniques known in the art.

In an alternative embodiment, where the ability of an IL-2 polypeptide fusion protein of the invention to multimerize is being evaluated, association with other components of the multimer can be assayed, e.g., by means well-known in the art, such as, for example, reducing and non-reducing gel chromatography, protein affinity chromatography, and affinity blotting. See generally, Phizicky et al., supra.

In addition, assays described herein (see Examples and Table 1) and otherwise known in the art may routinely be applied to measure the ability of IL-2 polypeptide fusion proteins of the present invention and fragments, variants and derivatives thereof to elicit biological activity and/or Therapeutic activity (either in vitro or in vivo) related to either the therapeutic protein portion and/or IL-2 polypeptide portion of the IL-2 polypeptide fusion protein of the present invention. Other methods will be known to the skilled artisan and are within the scope of the invention.

IL-2 polypeptide

As described above, an IL-2 polypeptide fusion protein of the invention comprises at least a fragment or variant of a therapeutic protein and at least a fragment or variant of human serum IL-2 polypeptide, which are associated with one another, preferably by genetic fusion or chemical conjugation.

The terms, human serum IL-2 polypeptide (HSA) and human IL-2 polypeptide (HA) are used interchangeably herein. The terms, “IL-2 polypeptide and “serum IL-2 polypeptide” are broader, and encompass human serum IL-2 polypeptide (and fragments and variants thereof) as well as IL-2 polypeptide from other species (and fragments and variants thereof).

As used herein, “IL-2 polypeptide” refers collectively to IL-2 polypeptide protein or amino acid sequence, or an IL-2 polypeptide fragment or variant, having one or more functional activities (e.g., biological activities) of IL-2 polypeptide. In particular, “IL-2 polypeptide” refers to human IL-2 polypeptide or fragments thereof (see EP 201 239, EP 322 094 WO 97/24445, WO95/23857) especially the mature form of human IL-2 polypeptide as shown in FIG. 15 and SEQ ID NO: 18, or IL-2 polypeptide from other vertebrates or fragments thereof, or analogs or variants of these molecules or fragments thereof.

In preferred embodiments, the human serum IL-2 polypeptide protein used in the IL-2 polypeptide fusion proteins of the invention contains one or both of the following sets of point mutations with reference to SEQ ID NO: 18: Leu-407 to Ala, Leu-408 to Val, Val-409 to Ala, and Arg-410 to Ala; or Arg-410 to A, Lys-413 to Gln, and Lys-414 to Gln (see, e.g., International Publication No. W095/23857, hereby incorporated in its entirety by reference herein). In even more preferred embodiments, IL-2 polypeptide fusion proteins of the invention that contain one or both of above-described sets of point mutations have improved stability/resistance to yeast Yap3p proteolytic cleavage, allowing increased production of recombinant IL-2 polypeptide fusion proteins expressed in yeast host cells.

As used herein, a portion of IL-2 polypeptide sufficient to prolong the therapeutic activity or shelf-life of the therapeutic protein refers to a portion of IL-2 polypeptide sufficient in length or structure to stabilize or prolong the therapeutic activity of the protein so that the shelf life of the therapeutic protein portion of the IL-2 polypeptide fusion protein is prolonged or extended compared to the shelf-life in the non-fusion state. The IL-2 polypeptide portion of the IL-2 polypeptide fusion proteins may comprise the full length of the HA sequence as described above or as shown in FIG. 15, or may include one or more fragments thereof that are capable of stabilizing or prolonging the therapeutic activity. Such fragments may be of 10 or more amino acids in length or may include about 15, 20, 25, 30, 50, or more contiguous amino acids from the HA sequence or may include part or all of specific domains of HA. For instance, one or more fragments of HA spanning the first two immunoglobulin-like domains may be used.

The IL-2 polypeptide portion of the IL-2 polypeptide fusion proteins of the invention may be a variant of normal HA. The therapeutic protein portion of the IL-2 polypeptide fusion proteins of the invention may also be variants of the therapeutic proteins as described herein. The term “variants” includes insertions, deletions and substitutions, either conservative or non conservative, where such changes do not substantially alter one or more of the oncotic, useful ligand-binding and non-immunogenic properties of IL-2 polypeptide, or the active site, or active domain which confers the therapeutic activities of the therapeutic proteins.

In particular, the IL-2 polypeptide fusion proteins of the invention may include naturally occurring polymorphic variants of human IL-2 polypeptide and fragments of human IL-2 polypeptide, for example those fragments disclosed in EP 322 094 (namely HA (Pn), where n is 369 to 419). The IL-2 polypeptide may be derived from any vertebrate, especially any mammal, for example human, cow, sheep, or pig. Non-mammalian IL-2 polypeptides include, but are not limited to, hen and salmon. The IL-2 polypeptide portion of the IL-2 polypeptide fusion protein may be from a different animal than the therapeutic protein portion.

Generally speaking, an HA fragment or variant will be at least 100 amino acids long, preferably at least 150 amino acids long. The HA variant may consist of or alternatively comprise at least one whole domain of HA, for example domains 1 (amino acids 1-194 of SEQ ID NO: 18), 2 (amino acids 195-387 of SEQ ID NO: 18), 3 (amino acids 388-585 of SEQ ID NO: 18), 1+2 (1-387 of SEQ ID NO: 18), 2+3 (195-585 of SEQ ID NO: 18). Each (amino acids 1-194 of SEQ ID NO: 18+amino acids 388-585 of SEQ ID NO: 18). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315 and Glu492 to Ala511.

Preferably, the IL-2 polypeptide portion of an IL-2 polypeptide fusion protein of the invention comprises at least one subdomain or domain of HA or conservative modifications thereof. If the fusion is based on subdomains, some or all of the adjacent linker is preferably used to link to the therapeutic protein moiety.

IL-2 Polypeptide Fusion Proteins

The present invention relates generally to IL-2 polypeptide fusion proteins and methods of treating, preventing, or ameliorating diseases or disorders. As used herein, “IL-2 polypeptide fusion protein” refers to a protein formed by the fusion of at least one molecule of IL-2 polypeptide (or a fragment or variant thereof) to at least one molecule of a therapeutic protein (or fragment or variant thereof). An IL-2 polypeptide fusion protein of the invention comprises at least a fragment or variant of a therapeutic protein and at least a fragment or variant of human serum IL-2 polypeptide, which are associated with one another, preferably by genetic fusion (i.e., the IL-2 polypeptide fusion protein is generated by translation of a nucleic acid in which a polynucleotide encoding all or a portion of a therapeutic protein is joined in-frame with a polynucleotide encoding all or a portion of IL-2 polypeptide) or chemical conjugation to one another. The therapeutic protein and IL-2 polypeptide protein, once part of the IL-2 polypeptide fusion protein, may be referred to as a “portion”, “region” or “moiety” of the IL-2 polypeptide fusion protein.

In one embodiment, the invention provides an IL-2 polypeptide fusion protein comprising, or alternatively consisting of, a therapeutic protein (e.g., as described in Table 1) and a serum IL-2 polypeptide protein. In other embodiments, the invention provides an IL-2 polypeptide fusion protein comprising, or alternatively consisting of, a biologically active and/or therapeutically active fragment of a therapeutic protein and a serum IL-2 polypeptide protein. In other embodiments, the invention provides an IL-2 polypeptide fusion protein comprising, or alternatively consisting of, a biologically active and/or therapeutically active variant of a therapeutic protein and a serum IL-2 polypeptide protein. In preferred embodiments, the serum IL-2 polypeptide protein component of the IL-2 polypeptide fusion protein is the mature portion of serum IL-2 polypeptide.

In further embodiments, the invention provides an IL-2 polypeptide fusion protein comprising, or alternatively consisting of, a therapeutic protein, and a biologically active and/or therapeutically active fragment of serum IL-2 polypeptide. In further embodiments, the invention provides an IL-2 polypeptide fusion protein comprising, or alternatively consisting of, a therapeutic protein and a biologically active and/or therapeutically active variant of serum IL-2 polypeptide. In preferred embodiments, the therapeutic protein portion of the IL-2 polypeptide fusion protein is the mature portion of the therapeutic protein.

In further embodiments, the invention provides an IL-2 polypeptide fusion protein comprising, or alternatively consisting of, a biologically active and/or therapeutically active fragment or variant of a therapeutic protein and a biologically active and/or therapeutically active fragment or variant of serum IL-2 polypeptide. In preferred embodiments, the invention provides an IL-2 polypeptide fusion protein comprising, or alternatively consisting of, the mature portion of a therapeutic protein and the mature portion of serum IL-2 polypeptide.

Preferably, the IL-2 polypeptide fusion protein comprises HA as the N-terminal portion, and a therapeutic protein as the C-terminal portion. Alternatively, an IL-2 polypeptide fusion protein comprising HA as the C-terminal portion, and a therapeutic protein as the N-terminal portion may also be used.

### EXAMPLES

### Example 1

### A Phase II Trial of Denileukin Diftitox—in Previously Treated, Advanced Non-Small Cell Lung Cancer (NSCLC)

Prior studies have shown that the interleukin-2 receptor (IL-2 r) is expressed on lung cancer cells suggesting that an IL-2 r targeted therapy may have a role in the management of NSCLC. Denileukin diftitox, ONTAK®, is a chimeric protein that targets the cytocidal properties of diphtheria toxin to cells that express IL-2 r and is approved for the treatment of cutaneous T-cell lymphoma. In this study, we evaluate the potential benefit of ONTAK in the treatment of advanced NSCLC. Methods: This multi-center phase II clinical trial is open for enrollment of patients with ECOG PS 0-2 and Stage IIIB/IV NSCLC who have failed at least one prior chemotherapy regimen. ONTAK is infused at 9 μg/kg on Day 1 of Cycle 1 only followed by 18 μg/kg/day Day 2-5 then every 21 days for a total of 6 cycles. Radiographic evaluation of tumor response is performed every 2 cycles. Results: Twenty patients have enrolled so far. There are 11 male and 9 female with a median age of 53 years (range 21-80). The mean number of prior treatment regimens is 2 (range 1-5). Patients received a mean of 3 (range 1-6) ONTAK cycles. There were no Grade 4 toxicities. Treatment-related Grade 3 toxicities included nausea (3), emesis (2), fever (2), fatigue (2), dehydration (2), edema (1), itching (1), pain at tumor site (1), flu-like symptoms (1), and headache (1). Symptomatic capillary leak syndrome and treatment-related death were not encountered. No patient was withdrawn due to treatment-related toxicity. Of the 20 patients enrolled, 4 are too early to evaluate for disease response, 2 had unconfirmed partial response using the RECIST criteria, 6 had stable disease, 7 had progressive disease, and 1 was not evaluable. The median time to disease progression fro all evaluable patients was 1.8 months (9 days-13 months) and the median overall survival was 4.3 months (2.7 weeks −15.5 months). Conclusion: Interim analysis of the first 20 patients enrolled in this trial suggests that ONTAK has promising activity and a favorable safety profile in the management of previously treated advanced NSCLC.

### EXAMPLE 2

Prior studies suggested the presence of interleukin-2 receptor (IL-2 r) in NSCLC. ONTAK(V is a chimeric protein that targets the cytocidal properties of diphtheria toxin to cells expressing IL-2 r. We evaluated the potential benefit of ONTAK in the treatment of advanced NSCLC.

Methods: This multi-center phase II trial enrolled patients (pts) with ECOG PS 0-2 and stage IIIB/IV NSCLC who progressed after at least one prior chemotherapy regimen. ONTAK is infused at 9 μg/kg on Day 1 of Cycle 1 only, followed by 18 μg/kg/day on Days 2-5, then every 21 days for a total of 6 cycles.

To be eligible for the study patients had to meet the following criteria: 1) Adult patient with a diagnosis of recurrent non-small cell lung cancer previously treated with surgery, and/or chemotherapy, and/or radiation therapy; 2) ECOG performance status of 0-2; 3) Adequate pre-treatment laboratory values; and 4) Measurable disease.

The protocol called for 6 treatments for all responders (CR+PR+SD). Response was defined as per RECIST criteria. In order to be evaluated, patients had to complete at least 2 cycles of treatment. ONTAK was infused at 9 mcg/kg on Day 1 of Cycle 1 only, followed by 18 mcg/kg/day on days 2-5, then every 21 days for a total of 6 cycles. Radiographic evaluation of tumor response was performed every 2 cycles. ONTAK therapy was discontinued in the presence of progressive disease or unacceptable toxicity. No concurrent chemotherapy, radiation therapy, or surgery was used during this trial.

Results: Forty patients were accrued with 23 males and a median age of 56 years (21-80). Mean number of prior chemotherapy regimens was 2 (1-5). Patients received a mean of 3 (1-6) cycles. Two (5%) are too early to evaluate, 2 (5%) had unconfirmed partial response by RECIST criteria, 14 (35%) had stable disease, 9 (23%) had progressive disease, and 13 (33%) were not evaluable due to clinical deterioration (8), toxicity (3), investigator's judgment (1) and voluntary withdrawal (1). The median time to disease progression for evaluable patients was 2.6 months (1-11 months). Overall survival was 4 months (0.2-15.6 months). One death from myocardial necrosis was attributed to treatment. Grade 3 toxicities (18) included GI, constitutional symptoms, 2 capillary leak syndrome (CLS) and one grade 4 CLS. Serial flow cytometry tests of peripheral blood lymphocytes on 11 consecutive patients showed decrease in CD8+25+ cells in all 5 pts with SD beyond 4 cycles compared to 2 out of 6 in patients with PD (P=0.06 by Exact Fisher Test). This study revealed disease control (PR+SD) of 40% in heavily pre-treated NSCLC.

What is claimed is:

1. A method for treating cancer in an individual, comprising administering an effective amount of an IL-2 fusion protein to the individual.

2. The method according to claim 1, wherein the cancer to be treated is a cancerous growth of breast, ovary, prostate, lung, pancreas, and colorectal cells.

3. The method according to claim 1, wherein the cancer to be treated is lung cancer

4. The method according to claim 3, wherein the lung cancer is refractory or relapsed.

5. The method according to claim 3, wherein the lung cancer comprises non-small cell lung cancer.

6. The method according to claim 3, where in the IL-2 fusion protein is a fusion protein comprising a therapeutic protein and an IL-2 polypeptide.

7. The method according to claim 3, where in the IL-2 fusion protein is a fusion between an IL-2 polypeptide or fragment and a toxin.

8. The method according to claim 7, where in the toxin is selected from the group consisting of cholera toxin, LT toxin, C3 toxin, Shiga toxin, Shiga-like toxin, ricin toxin, pertussis toxin, tetanus toxin, diphtheria toxin, and Pseudomonas exotoxin A.

9. The method according to claim 7, where in the toxin is diphtheria toxin.

10. The method according to claim 7, where in the IL-2 fusion protein is denileukin diftitox.

11. A method for treating cancer in an individual, comprising administering an effective amount of a hybrid molecule comprising at least a first part and a second part connected by covalent bonds, (a) wherein said first part comprises a portion of the binding domain of a cell-binding ligand effective to cause said hybrid molecule to bind to a cell of an animal; (b) wherein said second part comprises a therapeutic protein to be introduced into the cell.

12. The method of claim 11, wherein the portion of the binding domain of a cell-binding ligand binds to lymphocytes.

13. The method of claim 11, wherein the portion of the binding domain of a cell-binding ligand binds to the CD25 receptor on lymphocytes.

14. The method of claim 13, wherein the lymphocytes are immune suppressor cells.

15. The method of claim 11, wherein the lymphocytes are substantially inhibited or killed by the molecule.

16. The method of claim 15, wherein the lymphocytes are capable of infiltrating solid tumors.

17. The method of claim 16, wherein the lymphocytes are immune suppressor cells.

18. The method of claim 17, wherein the portion of the binding domain of a cell-binding ligand binds to IL-2 receptors on lymphocytes.

19. The method of claim 17, wherein the portion of the binding domain of a cell-binding ligand binds to soluble IL-2 receptors

20. A method for monitoring response to treatment in an individual with lung cancer, comprising administering an effective amount of IL-2 fusion protein to the individual and measuring soluble interleukin-2 receptors (sIL-2 R) levels of the individual.

21. The method according to claim 5, wherein the IL-2 fusion protein is denileukin diftitox.
